(19) United States
(12) Patent Application Publication
(10) Pub. No.: US 2021/0217511 A1
(43) Pub. Date: Jul. 15, 2021

(54) ADAPTIVE SCHEDULING FOR HEALTHCARE RESOURCES

(71) Applicant: International Business Machines Corporation, Armonk, NY (US)

(72) Inventors: Paul R. Bastide, Ashland, MA (US); Robert E. Loredo, North Miami Beach, FL (US); Matthew E. Broomhall, Goffstown, NH (US)

(21) Appl. No.: 16/739,571

(22) Filed: Jan. 10, 2020

(51) Int. Cl. G16H 40/20; G16H 10/60; G06Q 10/04; G06Q 10/06; G16H 15/00; G06F 40/174; G06F 40/186

(52) U.S. Cl. CPC G16H 40/20; G16H 10/60; G06Q 10/047; G06F 40/186; G16H 15/00; G06F 40/174; G06Q 10/06316

(57) ABSTRACT

Scheduling, by machine logic, the order of use of “medical resources” (see definition as set forth herein) during a scheduled medical visit by a patient to a “medical facility” (see definition as set forth herein) to have performed multiple “medical operations” (see definition as set forth herein). The scheduling is based on at least one of the following factors: distance between medical resources and availability, over time, of medical resources. Also, generating a “medical form user interface template” (see definition as set forth herein) that lists the scheduled medical operations in the order scheduled by the machine logic.

### BACKGROUND

Electronic health records (EHRs) contain the medical and treatment of patients—visits, prescriptions, lab orders, lab results and other clinical data—over the lifetime of each patient. This data enables primary care, emergency departments and specialists to track trends, derive insights and create custom treatment plans for each patient.

The concept of “medical operations” is known. Some medical operations relate to what are commonly called “medical examinations” (for example, having blood drawn), while other “medical operations” are more ancillary (for example, having hair shaved prior to a surgery, or making a co-pay for a medical examination at a medical office). As the term is used herein, a medical operation is any operation, process or procedure performed on, with or by a patient for medical purposes. Examples of “medical operations” include, without limitation: signing in for a medical appointment, signing out from a medical appointment, being fitted for medical equipment (for example, a prosthetic), a surgery, administration of a shot, drawing blood, doing a CAT scan, checking blood pressure, weighing a patient, doing a colonoscopy, etc.

It is known that a “medical operation” typically requires one, or more, “medical resources.” As the term “medical resources” is used herein, it does not refer to doctors, nurses or other human medical providers, but, rather, for the hardware needed to perform the medical operation. Examples of “medical resources” include, without limitation: area for signing in for a medical appointment, area for signing out from a medical appointment, room for being fitted for medical equipment (for example, a prosthetic), a medical device for fitting patients for medical equipment (for example, a Brannock Device used to fit patients for orthopedic footwear), a room and table for performing a surgery, a room and seat for administration of a shot, a room and rig for drawing blood, a room and machine for doing a CAT scan, an area and cuff device for checking blood pressure, an area with a scale for weighing a patient, etc. It is noted that “medical resources” generally are specific locations in a “medical facility” (that is, any structure, or set of structures, used primarily for medical purposes), or are associated with a specific location in a medical facility (for example, the hardware for performing a colonoscopy may be kept in a certain designated room in a hospital). For this reason, “medical resources” are treated herein as having specific locations, even if the medical resource is portable and/or could potentially be used in any room, or area, in the medical facility.

“Medical form user interface templates” are known. For purposes of this document, a medical form user interface template is any paper or computer-based form where medical data, obtained through performing medical operations is entered. An example of a paper based medical form user interface template is now set forth:
Patient Name: ______
Patient Age (in years): ______
Weight: ______
Blood pressure: ______
Patient check out time: ______

### SUMMARY

According to an aspect of the present invention, there is a method, computer program product and/or system that performs the following operations (not necessarily in the following order): (i) receiving a patient medical observation data set including information indicative of a plurality of medical operations to be performed on a patient and respectively corresponding plurality of medical resources needed to perform the plurality of medical operations; (ii) receiving a medical facility site map data set including location information respectively corresponding to each medical resource of the plurality of medical resources; (iii) determining, by machine logic, a plurality of orderings respectively indicating different orders in which the plurality of medical operations may be performed on the patient; (iv) for each given ordering of the plurality of orderings, determining an expected path length that the patient will have to travel to have performed on the patient the plurality of medical operations under the given ordering, with the expected path length being based on the medical facility site map data set; (v) selecting, by machine logic, a first ordering from the plurality of orderings based, at least in part, upon the expected path links respectively corresponding to the plurality of orderings; and (vi) performing the plurality of medical operations on the patient in an order according to the first ordering.

According to a further aspect of the present invention, there is a method, computer program product and/or system that performs the following operations (not necessarily in the following order): (i) receiving a patient medical observation data set including information indicative of a plurality of medical operations to be performed on a patient over the course of a scheduled medical appointment occurring on a given date and respectively corresponding plurality of medical resources needed to perform the plurality of medical operations; (ii) receiving medical resource availability data set including information indicative of availability of the plurality of medical resources during the scheduled medical appointment; (iii) determining, by machine logic, a plurality of orderings respectively indicating different orders in which the plurality of medical operations may be performed on the patient; (iv) for each given ordering of the plurality of orderings, determining availability of the medical resources; (v) selecting, by machine logic, a first ordering from the plurality of orderings based, at least in part, upon the availability of the medical resources as indicated by the medical resource availability data set; and (vi) performing the plurality of medical operations on the patient in an order according to the first ordering.

According to a further aspect of the present invention, there is a method, computer program product and/or system that performs the following operations (not necessarily in the following order): (i) receiving a patient medical observation data set including information indicative of a plurality of medical operations to be performed on a patient over the course of a scheduled medical appointment occurring on a given date and respectively corresponding plurality of medical resources needed to perform the plurality of medical operations; (ii) receiving a medical facility site map data set including location information respectively corresponding to each medical resource of the plurality of medical resources; (iii) receiving medical resource availability data set including information indicative of availability of the plurality of medical resources during the scheduled medical appointment; (iv) determining, by machine logic, a plurality of orderings respectively indicating different orders in which the plurality of medical operations may be performed on the patient; (v) for each given ordering of the plurality of orderings, determining an expected path length that the patient will have to travel to have performed on the patient the plurality of medical operations under the given ordering, with the expected path length being based on the medical facility site map data set; (vi) for each given ordering of the plurality of orderings, determining availability of the medical resources; (vii) selecting, by machine logic, a first ordering from the plurality of orderings based, at least in part, upon both of the following: (a) the expected path links respectively corresponding to the plurality of orderings, and (b) the availability of the medical resources as indicated by the medical resource availability data set; and (viii) performing the plurality of medical operations on the patient in an order according to the first ordering.

### BRIEF DESCRIPTION OF THE DRAWINGS

FIG. 1 is a block diagram view of a first embodiment of a system according to the present invention;

FIG. 2 is a flowchart showing a first embodiment method performed, at least in part, by the first embodiment system;

FIG. 3 is a block diagram showing a machine logic (for example, software) portion of the first embodiment system;

FIG. 4 is a plan view of a medical facility where the first embodiment system is in use;

FIG. 5 is a first screenshot of a patient form according to an embodiment of the present invention; and

FIG. 6 is a second screenshot of a patient form according to an embodiment of the present invention.

### DETAILED DESCRIPTION

Some embodiments of the present invention are directed to scheduling, by machine logic, the order of use of “medical resources” (see definition as set forth herein) during a scheduled medical visit by a patient to a “medical facility” (see definition as set forth herein) to have performed multiple “medical operations” (see definition as set forth herein). The scheduling is based on at least one of the following factors: distance between medical resources and availability, over time, of medical resources. Some embodiments are directed to generating a “medical form user interface template” (see definition as set forth herein) that lists the scheduled medical operations in the order scheduled by the machine logic. This Detailed Description section is divided into the following subsections: (i) The Hardware and Software Environment; (ii) Example Embodiment; (iii) Further Comments and/or Embodiments; and (iv) Definitions.

### I. The Hardware and Software Environment

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (for example, light pulses passing through a fiber-optic cable), or electrical signals transmitted through a wire.

A “storage device” is hereby defined to be anything made or adapted to store computer code in a manner so that the computer code can be accessed by a computer processor. A storage device typically includes a storage medium, which is the material in, or on, which the data of the computer code is stored. A single “storage device” may have: (i) multiple discrete portions that are spaced apart, or distributed (for example, a set of six solid state storage devices respectively located in six laptop computers that collectively store a single computer program); and/or (ii) may use multiple storage media (for example, a set of computer code that is partially stored in as magnetic domains in a computer's non-volatile storage and partially stored in a set of semiconductor switches in the computer's volatile memory). The term “storage medium” should be construed to cover situations where multiple different types of storage media are used.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

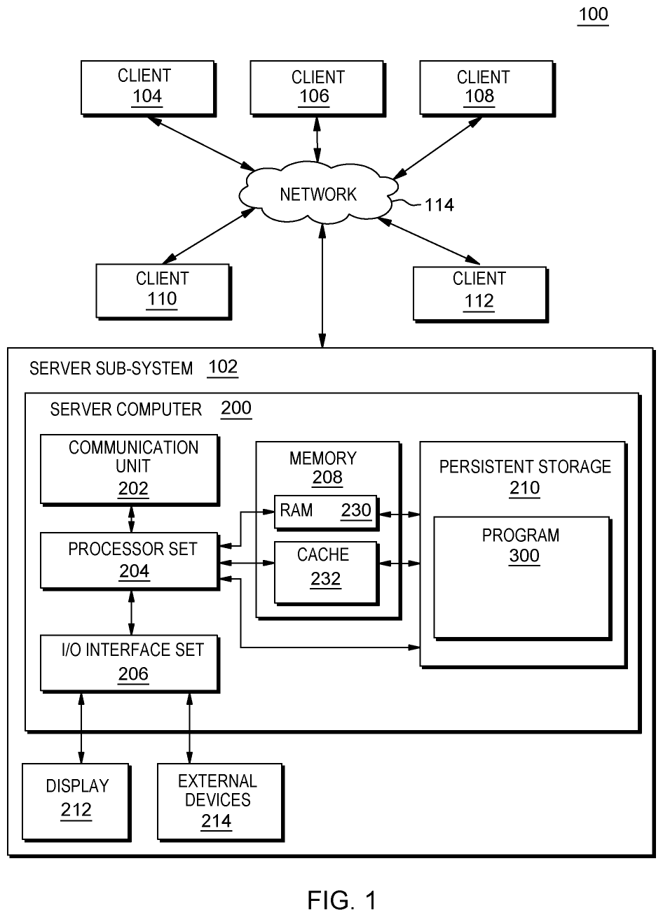

As shown in FIG. 1, networked computers system 100 is an embodiment of a hardware and software environment for use with various embodiments of the present invention. Networked computers system 100 includes: server subsystem 102 (sometimes herein referred to, more simply, as subsystem 102); client subsystems 104, 106, 108, 110, 112; and communication network 114. Server subsystem 102 includes: server computer 200; communication unit 202; processor set 204; input/output (I/O) interface set 206; memory 208; persistent storage 210; display 212; external device(s) 214; random access memory (RAM) 230; cache 232; and program 300.

Subsystem 102 may be a laptop computer, tablet computer, netbook computer, personal computer (PC), a desktop computer, a personal digital assistant (PDA), a smart phone, or any other type of computer (see definition of “computer” in Definitions section, below). Program 300 is a collection of machine-readable instructions and/or data that is used to create, manage and control certain software functions that will be discussed in detail, below, in the Example Embodiment subsection of this Detailed Description section.

Subsystem 102 is capable of communicating with other computer subsystems via communication network 114. Network 114 can be, for example, a local area network (LAN), a wide area network (WAN) such as the Internet, or a combination of the two, and can include wired, wireless, or fiber optic connections. In general, network 114 can be any combination of connections and protocols that will support communications between server and client subsystems.

Subsystem 102 is shown as a block diagram with many double arrows. These double arrows (no separate reference numerals) represent a communications fabric, which provides communications between various components of subsystem 102. This communications fabric can be implemented with any architecture designed for passing data and/or control information between processors (such as microprocessors, communications and network processors, etc.), system memory, peripheral devices, and any other hardware components within a computer system. For example, the communications fabric can be implemented, at least in part, with one or more buses.

Memory 208 and persistent storage 210 are computer-readable storage media. In general, memory 208 can include any suitable volatile or non-volatile computer-readable storage media. It is further noted that, now and/or in the near future: (i) external device(s) 214 may be able to supply, some or all, memory for subsystem 102; and/or (ii) devices external to subsystem 102 may be able to provide memory for subsystem 102. Both memory 208 and persistent storage 210: (i) store data in a manner that is less transient than a signal in transit; and (ii) store data on a tangible medium (such as magnetic or optical domains). In this embodiment, memory 208 is volatile storage, while persistent storage 210 provides nonvolatile storage. The media used by persistent storage 210 may also be removable. For example, a removable hard drive may be used for persistent storage 210. Other examples include optical and magnetic disks, thumb drives, and smart cards that are inserted into a drive for transfer onto another computer-readable storage medium that is also part of persistent storage 210.

Communications unit 202 provides for communications with other data processing systems or devices external to subsystem 102. In these examples, communications unit 202 includes one or more network interface cards. Communications unit 202 may provide communications through the use of either or both physical and wireless communications links. Any software modules discussed herein may be downloaded to a persistent storage device (such as persistent storage 210) through a communications unit (such as communications unit 202).

I/O interface set 206 allows for input and output of data with other devices that may be connected locally in data communication with server computer 200. For example, I/O interface set 206 provides a connection to external device set 214. External device set 214 will typically include devices such as a keyboard, keypad, a touch screen, and/or some other suitable input device. External device set 214 can also include portable computer-readable storage media such as, for example, thumb drives, portable optical or magnetic disks, and memory cards. Software and data used to practice embodiments of the present invention, for example, program 300, can be stored on such portable computer-readable storage media. I/O interface set 206 also connects in data communication with display 212. Display 212 is a display device that provides a mechanism to display data to a user and may be, for example, a computer monitor or a smart phone display screen.

In this embodiment, program 300 is stored in persistent storage 210 for access and/or execution by one or more computer processors of processor set 204, usually through one or more memories of memory 208. It will be understood by those of skill in the art that program 300 may be stored in a more highly distributed manner during its run time and/or when it is not running. Program 300 may include both machine readable and performable instructions and/or substantive data (that is, the type of data stored in a database). In this particular embodiment, persistent storage 210 includes a magnetic hard disk drive. To name some possible variations, persistent storage 210 may include a solid-state hard drive, a semiconductor storage device, read-only memory (ROM), erasable programmable read-only memory (EPROM), flash memory, or any other computer-readable storage media that is capable of storing program instructions or digital information.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

### II. Example Embodiment

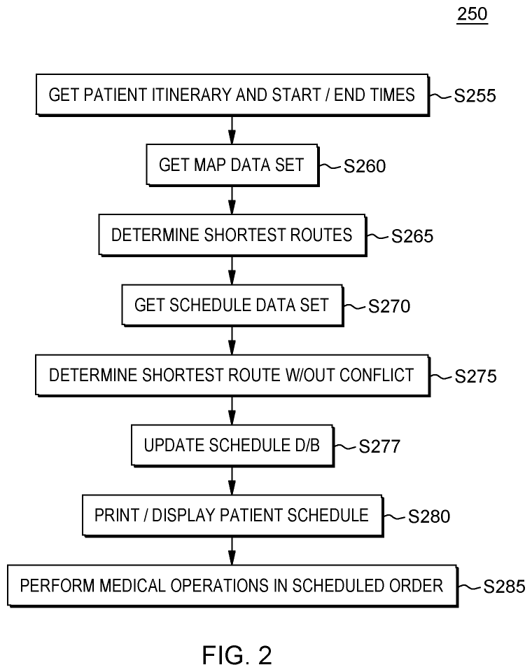

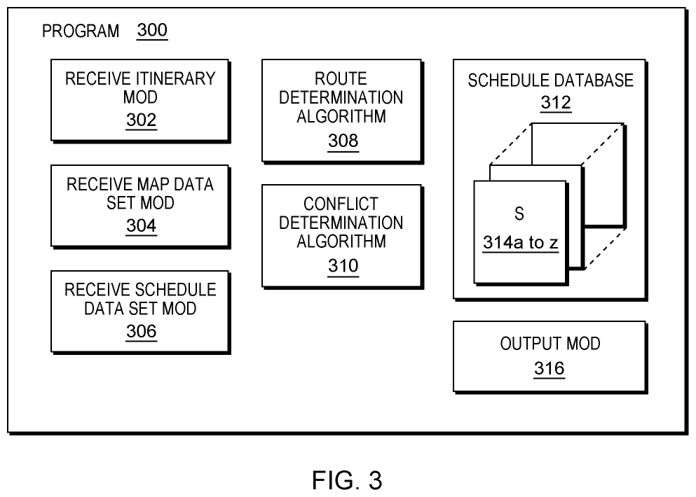

As shown in FIG. 1, networked computers system 100 is an environment in which an example method according to the present invention can be performed. As shown in FIG. 2, flowchart 250 shows an example method according to the present invention. As shown in FIG. 3, program 300 performs or controls performance of at least some of the method operations of flowchart 250. This method and associated software will now be discussed, over the course of the following paragraphs, with extensive reference to the blocks of FIGS. 1, 2 and 3.

Processing begins at operation S255, where receive itinerary module (“mod”) 302 receives an itinerary for a scheduled patient visit. In this example, the itinerary data is received from client device 104 (that is, the computer used by the scheduling office associated with the medical facility) over communication network 114.

In this example the itinerary includes: (i) scheduled start time for the patient visit; (ii) scheduled and time for the patient visit; and (iii) the “medical operations” to be performed during the patient visit (and any limitations on the order in which the medical operations must, or may, be performed). In this example, the medical operations of the itinerary are as follows: (i) check in with reception; (ii) have a vision test; (iii) get teeth cleaned; (iv) draw blood; and (v) check out with a discharge desk. In this example, the only limitations on the order of the operation are that the check in must be done first and the check out must be done last. In different embodiments, different operations may be treated as medical operations that are included in the itinerary. For example, as an alternative, check in and check out may not be considered as medical operations to be listed on the itinerary, especially because their place within the ordering of medical operations is fixed and immutable under the rules. As a further example, the simple operation like weighing the patient might not be treated as a medical operation because it is so quick that it does not affect schedule, and the scale is easily portable, so it does not affect patient transport through the medical facility. In other words, not every medical related thing that happens to the patient necessarily needs to be considered as one of the “medical operations.” On the other hand, things that affect scheduling and/or transport should typically be considered as “medical operations.”

Processing proceeds to operation S260, where receive map data set mod 304 receives a map data set that includes information about where the various medical operations are performed within the space of the medical facility. Sometimes this data may be relatively unchanging. For example, a hospital may have all surgeries performed in a certain room which does not change often because of the fixtures required to perform surgery. Other times this data may change frequently. For example, a hospital may have one room for the phlebotomist that draws blood on Mondays, Wednesdays and Fridays and another room for the phlebotomist that draws blood on Tuesdays and Thursdays. The map data set should include mapping data that covers at least the time for which the patient visit is being scheduled. In this example, the map data set is sent by client device 106 (the computer used by the facilities department) through communication network 114 to mod 304. In this example, the map data set is received approximately one hour before the scheduled patient visit, but this timing may vary depending on the specific application.

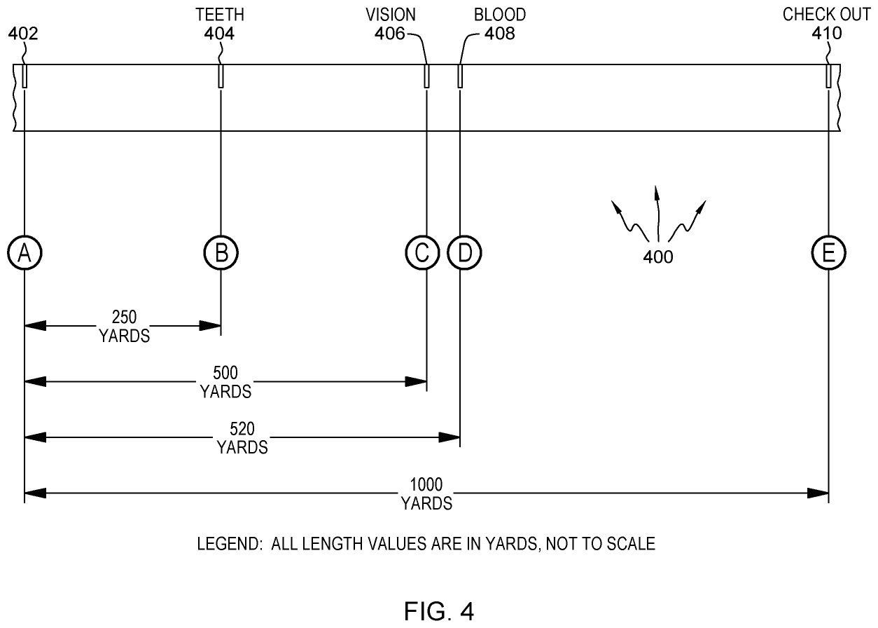

As shown in FIG. 4, in this example medical facility 400 includes: room 402, room 404, room 406, room 408 and room 410. For the time of the patient visit the mapping of the map data set received at operation S260 is as follows: (i) room 402 is used for patient visit check in; (ii) room 404 is used for teeth cleaning; (iii) room 406 is used for vision tests; (iv) room 408 is used for drawing blood; and (v) room 410 is used for check out.

Processing proceeds to operation S265, route determination algorithm determines the three shortest routes that the patient can potentially use during the medical visit, while having all the required medical operations performed, under any restrictions on ordering that may exist.

In this example, the shortest route for the patient through medical facility 400 is determined to be as follows: (i) check in at room 402 (location A); (ii) teeth cleaning at room 404 (location B); (iii) vision test at room 406 (location C); (iv) blood draw at room 408 (location D); and (v) check out at room 410 (location E). As can be seen from the dimensions given in FIG. 4, this route is 1000 yards long.

In this example, the second shortest route for the patient through medical facility 400 is determined to be as follows: (i) check in at room 402 (location A); (ii) teeth cleaning at room 404 (location B); (iii) blood draw at room 408 (location D); (iv) vision test at room 406 (location C); and (v) check out at room 410 (location E). As can be seen from the dimensions given in FIG. 4, this route is 1040 yards long.

In this example, the third shortest route for the patient through medical facility 400 is determined to be as follows: (i) check in at room 402 (location A); (ii) vision test at room 406 (location C); (iii) teeth cleaning at room 404 (location B); (iv) blood draw at room 408 (location D); and (v) check out at room 410 (location E). As can be seen from the dimensions given in FIG. 4, this route is 1520 yards long.

In embodiments of the present invention that only consider patient travel distance, the shortest route (described 3 paragraphs previous) would be used to schedule the medical operations during the patient visit. However, this embodiment also takes into account “medical resource” availability, so further processing must be performed before the patient's schedule is actually determined.

Processing proceeds to operation S270, where receive schedule data set mod 306 receives schedules for other patients' use of the medical resources, during the time of the patient visit, from schedule database 312. Each patient's schedule is stored as one of the records 314a to 314z in database 312.

Processing proceeds to operation S275, where conflict determination algorithm 310 determines the shortest route such that there are no scheduling conflicts with other patients for any of the medical resources to be used during the patient's visit. In this example, it is determined that, under the shortest patient route (that is, the 1000 yard route), there would be a medical resource conflict with another patient at the following medical resource: vision testing room 406. Therefore, this route, and associated patient's schedule, is rejected even though it is the shortest travel distance. It is further determined that the second shortest patient route (that is, the 1040 yard route) does not lead to any conflicts with other patients. Therefore, this route (specifically room 402 then 404 then 408 then 406 then 410) is determined to be the patient's schedule for the patient scheduled visit, and the order of the medical operations to be performed on the patient at the patient scheduled visit is now effectively determined as: check in, then teeth cleaning, then blood draw, then vision test, and, finally, check out.

Processing proceeds to operation S277, where output mod 316 adds the patient's schedule as one of the records 314a to 314z of schedule database 312, so that conflicts with other patients, to be scheduled at a later time, can be detected.

Processing proceeds to operation S280 where output mod 316: (i) prints a hard copy of a medical form user interface template for the patient visit; (ii) sends a soft copy of the medical form user interface template for the patient visit through communication network 114 to client device 108 (that is, a tablet computer used by a medical employee tasked with shepherding the patient through her appointment at medical facility 400). In this example, the medical form user interface template is as follows:
Patient: George Washington
Check in time: ______
Issues discovered during oral examination: ______
Amount of blood drawn: ______
Patient vision heuristic: ______
Check out time: ______
   Notice that the medical form user interface template orders the fields to be filled in in the order in which the corresponding medical operations are performed upon the patient. This can help the person, or persons, filling information into the form to put the correct information into the correct field. In other words, the form will be filled out in top to bottom order during the patient visit, which is highly intuitive and user-friendly.

Processing proceeds to operation S285, where the patient shows up for the appointment at medical facility 400 and the medical operations are performed on the patient's in the order determined by program 300. While this embodiment determines the patient's schedule considering only patient travel distance and medical resource availability, other factors may be considered such as: elevators, crowded hallways, cumulative patient fatigue/discomfort, the least patient mobility effort, the least provider mobility effort, the fewest provider contacts, and the infection rate related to observed issues.

### III. Further Comments and/or Embodiments

Some embodiments of the present invention recognize the following facts, potential problems and/or potential areas for improvement with respect to the current state of the art: (i) the patient's EHR (electronic health records) are only as good as the data that is captured and this information capture must balance speed, accuracy and precision; (ii) many hospitals and practices struggle to manage the capture observations efficiently (for example, height, weight, and blood draws); (iii) often, the provider is unable to use the medical resource (for example, a piece of medical equipment) needed due to another patient's engagement with the resource (for example, a CT (computed tomography) scan is in use by another patient); and (iv) electronic medical records (EMRs), while generally scoped to a practice or medical site also suffer this problem.

Some embodiments of the present invention include the following scenario: (i) Alice is a patient of Nurse Bob; (ii) Nurse Bob loads Alice's medical form; (iii) Nurse Bob leads Alice from the waiting area to the observation room; (iv) Nurse Bob is reviewing the first observational entry (for example, blood pressure); (v) Nurse Bob walks right by the scales; and (vi) there is a need to enable healthcare personnel to adapt to the locations of healthcare professions, patients and measurement devices to optimize the patient experience and minimize the overhead of forms.

Some embodiments of the present invention may include one, or more, of the following operations, features, characteristics and/or advantages: (i) re-orders, or sets a custom order, for medical operations on a medical form to make it faster to collect and/or enter the medical data into the medical form; (ii) extends the utility of tracking patients and resources so that observational data is captured quickly and efficiently; and (iii) geo-fences hospital resources in order to push changes in the forms and data entry/capture.

As an example of item (iii), in the list of the previous paragraph (sometimes herein referred to as the geofencing feature), a portal device may be able to be moved on a floor, and each floor may have it so a geofence is a specific area on the map where a resource is available. This can help with mapping “medical resources” to physical locations at a medical facility.

For a given patient, an embodiment of method for entering data into a form by healthcare professionals includes the following operations: (a) identifying the form the provider needs to fill out for the patient, (b) extracting the observational data the provider needs to capture, (c) retrieving attributes of the provider and resources involved in filling out the form including: (1) preferences based on a plurality of providers' path to engage the patient, (2) path traveled by provider and patient, (3) optimistic testing needs (comorbidities), and (4) availability, (d) calculating the path through the form to maximize the patient and healthcare provider encounter, (e) adapting, based on the calculated path, the grouping of tests and form entry, (f) includes geo-fenced push notifications (that is, the resources may be geo-fenced), (g) considering the providers/clinicians activity while load balancing multiple patients, such as a nurse in charge of twelve patients, and (h) recalculating the flow on demand and maintain a shortest path graph in memory to more easily adapt.

Some embodiments of the present invention may include one, or more, of the following operations, features, characteristics and/or advantages: (i) scoped to single form panel or a series of dialogs (for example, page 1 of 7); (ii) improves patient care through minimizing transitions during a patient-provider encounter; (iii) speeds EHR data entry and reduces contention with observation devices for example, (start or end of an encounter); (iv) provides efficient mapping of resources for patients-providers during data entry; (v) can be implemented an addition to existing medical software products; and/or (vi) expands generalized form entry usage beyond medical/healthcare.

The following scenario shows one way in which an embodiment of the present invention may be efficiently employed: (i) Alice is a patient of nurse Bob; (ii) nurse Bob loads Alice's medical form; and (iii) Alice's patient details and longitudinal patient history are loaded.

As an example of the above:

Name: Alice Patient

Gender: Female Age: 73

Phone: 9999-9999-9999

Address: 99 Main Street, Boston, Mass. 0101010

Symptoms:
1. Scratchy Throat
2. Prior Indications of Fever, Chills
3. Producing mucus

Prior History:
1. Bronchitis
2. Viral Pneumonia

Some embodiments of the present invention extract the details from history as is shown in the following sample data:

[Throat]->Infection

[Fever, Chills]->Weight Fluctuations

[Throat|Fever|Chills]->O2 Levels, Temperature

[Produc*mucus]->Observation of Pulmonary System

[Bronchitis|*Pneumonia*] & [!over x-ray limit]->Pulmonary X-Ray

Some embodiments of the present invention associate the observation details the provider needs to capture with forms, and identifies potential medical operations (in this example, called “observations”) that need to be performed on the patient during a visit to a medical facility:

Observation: Weight

Observation: Temperature

Observation: O2 Levels

Observation: Throat

Observation: Pulmonary System X-Ray

An example of attributes of the provider and resources involved in filling out the form is now set forth: (i) Nurse Bob wants to quickly capture Weight/Temperature; (ii) Nurse Bob wants early analysis of Throat; (iii) X-Ray is available for two (2) hours; (iv) Path: Waiting Room to Observation Room 1; and (v) Resources passed include X-Ray and Scale. In this example, machine logic of the present invention determine the best order to perform the medical operations using a custom ordering of the form to guide the medical professional(s) in performing the medical operations and, concomitantly entering the medical data (that is, the “observations”) into the form.

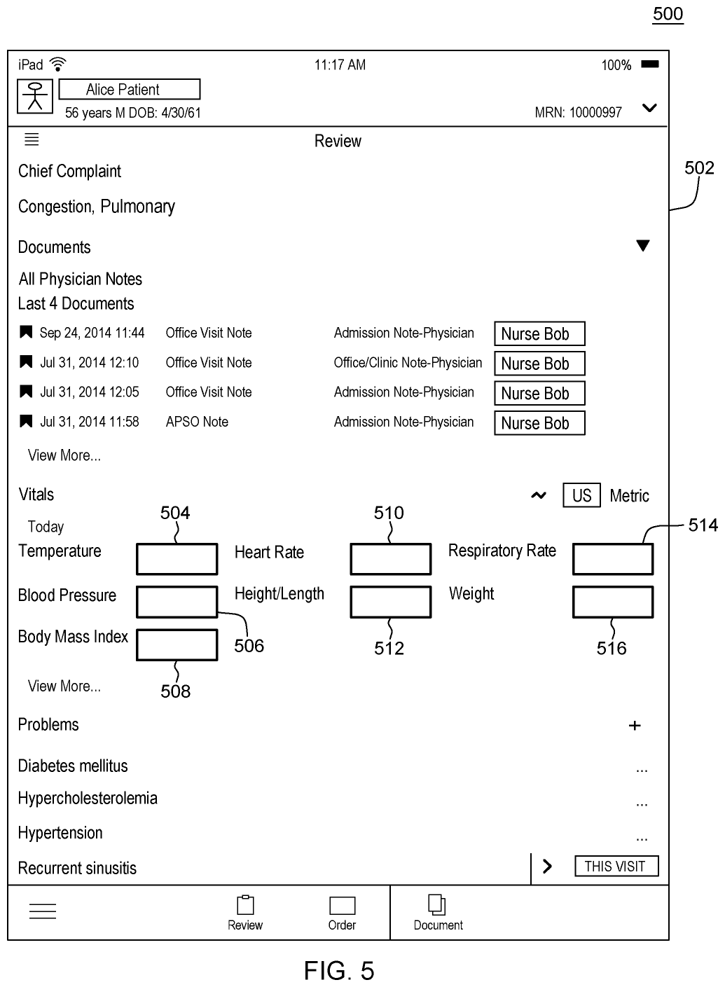

FIG. 5, diagram 500, is a first patient form according to an embodiment of the present invention and includes the following patient information: patient data form 502; temperature block 504, blood pressure block 506, body mass index block 508; heart rate block 510; height/length block 512; respiratory rate block 514; and weight block 516.

Some embodiments of the present invention may include one, or more, of the following operations, features, characteristics and/or advantages: (i) calculates the path through the form to maximize the patient and healthcare provider encounter, for example: weight, body mass index, blood draw, observation and x-ray; and (ii) adapts, based on the calculated path, the grouping of tests and form entry.

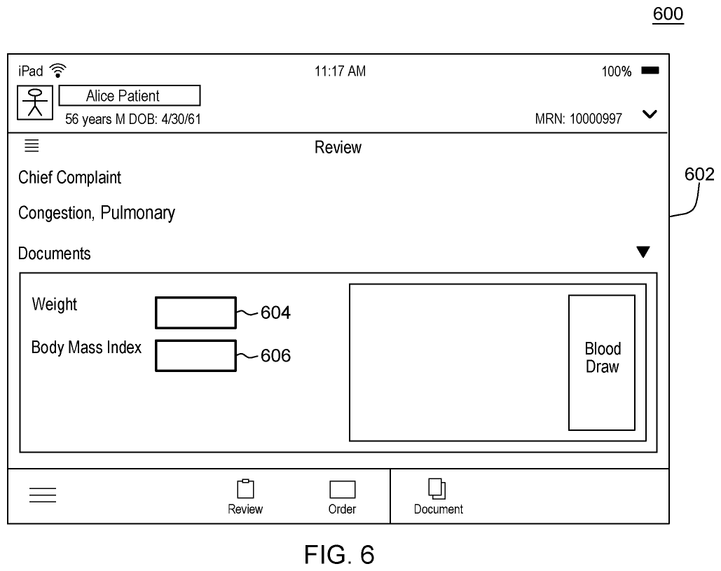

FIG. 6, diagram 600, is a second patient form according to an embodiment of the present invention and includes the following patient information: patient data form 602; weight block 604; and body mass index block 606.

Some embodiments of the present invention may include one, or more, of the following operations, features, characteristics and/or advantages: (i) nurse Bob leads from the waiting area to the scale; (ii) based on the captured data, recalculates the path through the form to maximize the patient and healthcare provider encounter; (iii) nurse Bob leads Alice to the blood draw; (iv) integrates with any EHR or EMR (electronic medical records) or other clinician systems, for example, Epic, Cerner, ReDoc; (v) identifies the form the provider needs to fill out for the patient; (vi) loads the patient specific data, longitudinal patient history/record which includes data on diseases, symptoms and prior observational data captured; (vii) preloads forms related to a specific patient; (viii) pre-analyzes the potential observational data, such as weight, height, blood pressure, sit-to-stand test; and (ix) considers the preferences of the clinician, specialist or provider, for example, always get a blood sample, ocular observation and includes: (a) preferences based on a plurality of providers' path to engage the patient, (b) path traveled by provider and patient, (c) optimistic testing needs (comorbidities), and (d) availability; and (x) considers additional observations based on cohort risks, for example, over 50, male, risk of specific disease.

Some embodiments of the present invention may include one, or more, of the following operations, features, characteristics and/or advantages: (i) extracting the observational data the provider needs to capture; (ii) extracts, from the observational, data the clinician needs to capture; (iii) uses medical NLP (neuro-linguistic programming) to determine an associated observation based on passed medical history or chief complaint; (iv) retrieves attributes of the provider and resources involved in the filling out the form (based on the attributes extracted from the form, and the given location on the form (Z-Order), such as observed weight); (v) the resource (scale) is associated with weight observation; (vi) determining that the location of the scale is located at x, y; (vii) locations are determined for each form element; (viii) calculates the path through the form to maximize the patient and healthcare provider encounter; (ix) weights the ordering of the form with Z-Order, based on location and the likelihood of needing an additional element in the dialog; (x) adapts, based on the calculated path, the grouping of tests and form entry; (xi) adapts the path based on the Z-Order and location for the form entry; and (xii) additionally considers providers/clinicians activity while load balancing multiple patients, for example, a nurse in charge of twelve patients.

A method for optimizing healthcare data entry by healthcare professionals for patients according to an embodiment of the present invention includes the following operations (not necessarily in the following order): (i) identifies information to be collected by a healthcare professional to complete data entry for at least one patient; (ii) analyzes the information to identify resources and availability of the resources; (iii) calculates an order to collect information for a patient based on the availability of the resources and optimization criteria; (iv) follows the calculated order for the patient related data entry; (v) adjusts the form dynamically based on the collected information and changes to the availability of the resources; (vi) identifies information to be collected is on the forms and the calculated order allows for the entries to be intermingled between a first form and a second form; and (vii) the optimization criteria is based on a current condition of the patient and a minimization of data collection time.

Some embodiments of the present invention may include one, or more, of the following operations, features, characteristics and/or advantages: (i) aims to adapt the forms, based on the pathway to the apparatus and devices (for example, the weight is the first thing in the specific office for the patient-clinician and therefore it should be at the top of the screen; and (ii) adapts the forms as a clinician engages with the patient such that a top-down flow happens in a form as the apparatuses are available; and/or (iii) identifies a set of patient encounter forms and transforms the elements across a form into a useful order such that an availability of resources dictates the flows of the form as such the forms' order across each page is dictated by the availability and path travelled.

Item (iii) in the list of the previous paragraph will now be discussed. The path taken in the hospital for each user is unique, constrained by the availability of medical resources. Entering a patient's name, skipping halfway down a page, and entering blood pressure can potentially lead to inconvenience and even errors (which errors can, of course, have drastic consequences in a medical context. Adaptively reorganizing the page so patient's name, then the blood pressure, and the next closest and available medical resource is presented.

### IV. Definitions

Present invention: should not be taken as an absolute indication that the subject matter described by the term “present invention” is covered by either the claims as they are filed, or by the claims that may eventually issue after patent prosecution; while the term “present invention” is used to help the reader to get a general feel for which disclosures herein are believed to potentially be new, this understanding, as indicated by use of the term “present invention,” is tentative and provisional and subject to change over the course of patent prosecution as relevant information is developed and as the claims are potentially amended.

Embodiment: see definition of “present invention” above—similar cautions apply to the term “embodiment.”

and/or: inclusive or; for example, A, B “and/or” C means that at least one of A or B or C is true and applicable.

Including/include/includes: unless otherwise explicitly noted, means “including but not necessarily limited to.”

Module/Sub-Module: any set of hardware, firmware and/or software that operatively works to do some kind of function, without regard to whether the module is: (i) in a single local proximity; (ii) distributed over a wide area; (iii) in a single proximity within a larger piece of software code; (iv) located within a single piece of software code; (v) located in a single storage device, memory or medium; (vi) mechanically connected; (vii) electrically connected; and/or (viii) connected in data communication.

Computer: any device with significant data processing and/or machine readable instruction reading capabilities including, but not limited to: desktop computers, mainframe computers, laptop computers, field-programmable gate array (FPGA) based devices, smart phones, personal digital assistants (PDAs), body-mounted or inserted computers, embedded device style computers, application-specific integrated circuit (ASIC) based devices.

What is claimed is:

1. A computer-implemented method (CIM) comprising:
receiving a patient medical observation data set including information indicative of a plurality of medical operations to be performed on a patient and respectively corresponding plurality of medical resources needed to perform the plurality of medical operations;
receiving a medical facility site map data set including location information respectively corresponding to each medical resource of the plurality of medical resources;
determining, by machine logic, a plurality of orderings respectively indicating different orders in which the plurality of medical operations may be performed on the patient;
for each given ordering of the plurality of orderings, determining an expected path length that the patient will have to travel to have performed on the patient the plurality of medical operations under the given ordering, with the expected path length being based on the medical facility site map data set;
selecting, by machine logic, a first ordering from the plurality of orderings based, at least in part, upon the expected path links respectively corresponding to the plurality of orderings; and
performing the plurality of medical operations on the patient in an order according to the first ordering.

2. The CIM of claim 1 further comprising:
generating a computer displayable medical form user interface template designed to facilitate data entry for data obtained during each medical operation of the plurality of medical operations, with the plurality of medical operations being presented on the medical form user interface template in an order according to the first ordering.

3. The CIM of claim 2 further comprising:
prior to the performance of the plurality of medical operations on the patient, displaying the medical form user interface template on a display device.

4. The CIM of claim 1 wherein all of the medical resources of the plurality of medical resources are medical machines respectively having relatively permanent locations within the medical facility.

5. The CIM of claim 1 wherein a first medical resources of the plurality of medical resources is medical machines that is portable and varies in location within the medical facility.

6. The CIM of claim 1 wherein the first ordering corresponds to the smallest path length.

7. A computer-implemented method (CIM) comprising:
receiving a patient medical observation data set including information indicative of a plurality of medical operations to be performed on a patient over the course of a scheduled medical appointment occurring on a given date and respectively corresponding plurality of medical resources needed to perform the plurality of medical operations;
receiving medical resource availability data set including information indicative of availability of the plurality of medical resources during the scheduled medical appointment;
determining, by machine logic, a plurality of orderings respectively indicating different orders in which the plurality of medical operations may be performed on the patient;
for each given ordering of the plurality of orderings, determining availability of the medical resources;
selecting, by machine logic, a first ordering from the plurality of orderings based, at least in part, upon the availability of the medical resources as indicated by the medical resource availability data set; and
performing the plurality of medical operations on the patient in an order according to the first ordering.

8. The CIM of claim 7 further comprising:
generating a computer displayable medical form user interface template designed to facilitate data entry for data obtained during each medical operation of the plurality of medical operations, with the plurality of medical operations being presented on the medical form user interface template in an order according to the first ordering.

9. The CIM of claim 8 further comprising:
prior to the performance of the plurality of medical operations on the patient, displaying the medical form user interface template on a display device.

10. The CIM of claim 7 wherein all of the medical resources of the plurality of medical resources are medical machines respectively having relatively permanent locations within the medical facility.

11. The CIM of claim 7 wherein a first medical resources of the plurality of medical resources is medical machines that is portable and varies in location within the medical facility.

12. The CIM of claim 7 wherein the first ordering corresponds to an ordering where there is no conflict between patients for any medical resource of the plurality of medical resources.

13. A computer-implemented method (CIM) comprising:
receiving a patient medical observation data set including information indicative of a plurality of medical operations to be performed on a patient over the course of a scheduled medical appointment occurring on a given date and respectively corresponding plurality of medical resources needed to perform the plurality of medical operations;
receiving a medical facility site map data set including location information respectively corresponding to each medical resource of the plurality of medical resources;
receiving medical resource availability data set including information indicative of availability of the plurality of medical resources during the scheduled medical appointment;
determining, by machine logic, a plurality of orderings respectively indicating different orders in which the plurality of medical operations may be performed on the patient;
for each given ordering of the plurality of orderings, determining an expected path length that the patient will have to travel to have performed on the patient the plurality of medical operations under the given ordering, with the expected path length being based on the medical facility site map data set;
for each given ordering of the plurality of orderings, determining availability of the medical resources;
selecting, by machine logic, a first ordering from the plurality of orderings based, at least in part, upon both of the following: (i) the expected path links respectively corresponding to the plurality of orderings, and (ii) the availability of the medical resources as indicated by the medical resource availability data set; and
performing the plurality of medical operations on the patient in an order according to the first ordering.

14. The CIM of claim 13 further comprising:
generating a computer displayable medical form user interface template designed to facilitate data entry for data obtained during each medical operation of the plurality of medical operations, with the plurality of medical operations being presented on the medical form user interface template in an order according to the first ordering.

15. The CIM of claim 14 further comprising:
prior to the performance of the plurality of medical operations on the patient, displaying the medical form user interface template on a display device.

16. The CIM of claim 13 wherein all of the medical resources of the plurality of medical resources are medical machines respectively having relatively permanent locations within the medical facility.

17. The CIM of claim 13 wherein a first medical resources of the plurality of medical resources is medical machines that is portable and varies in location within the medical facility.

18. The CIM of claim 13 wherein the first ordering corresponds to the smallest path length where there is no conflict between patients for any medical resource of the plurality of medical resources.
